(19) United States
(12) Patent Application Publication
(10) Pub. No.: US 2007/0122343 A1
(43) Pub. Date: May 31, 2007

(54) Non-invasive assay for the assessment of functioning and/or structure of the gut

(76) Inventor: Ross Norman Butler, Unley (AU)

Correspondence Address: THE MCCALLUM LAW FIRM, P. C., 685 BRIGGS STREET, PO BOX 929, ERIE, CO, 80516, US

(21) Appl. No.: 10/557,221

(22) PCT Filed: May 14, 2004
(86) PCT No.: PCT/AU04/00640

§ 371 Date: Jan. 29, 2007

(30) Foreign Application Priority Data
May 16, 2003 (AU) 2003902390

(51) Int. Cl. A61K 49/00

(52) U.S. Cl. 424/9.1

(57) ABSTRACT

The present invention relates to a diagnostic method for assessing the functioning and/or structural integrity of the gut in animal subjects. The method of the present invention is predicated on assessing the absorption and/or metabolism of one or more test sugars or functional equivalents such as phosphorylated sugars, oligopeptides or oligonucleotides in an animal subject, wherein the level and/or rate of absorption or metabolism of each molecule is examined to give an overall indication of gut functioning and/or gut structural integrity in the animal subject. The methods of the present invention have inter alia medical and diagnostic applications in humans, such as for the diagnosis of various pathologies associated with changes in gut function or permeability. Furthermore, the methods of the present invention may also be applied to assess gut function or structural integrity in other animal, including avian, species.

### BACKGROUND OF THE INVENTION

1. Field of the Invention

The present invention relates to a diagnostic method for assessing the functioning and/or structural integrity of the gut in animal subjects. The method of the present invention is predicated on assessing the absorption and/or metabolism of one or more test sugars or functional equivalents such as phosphorylated sugars, oligopeptides or oligonucleotides in an animal subject, wherein the level and/or rate of absorption or metabolism of each molecule is examined to give an overall indication of gut functioning and/or gut structural integrity in the animal subject. The methods of the present invention have inter alia medical and diagnostic applications in humans, such as for the diagnosis of various pathologies associated with changes in gut function or permeability. Furthermore, the methods of the present invention may also be applied to assess gut function or structural integrity in other animal, including avian, species.

2. Description of the Prior Art

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

Dual sugar permeability tests have been described to measure leakiness of the small intestine. This commonly involves the use of a disaccharide and particularly what might be considered an unreactive disaccharide that is not susceptible to enzymatic cleavage or active transport or the small intestine. Typically lactulose is used. A monosaccharide is also used and the two commonly used monosaccharides for this purpose are rhamnose or mannitol, these are chosen at least in part because their presence is not masked during testing by other metabolites commonly present in a body. Some time after injestion of the sugars their presence in blood is tested, either directly or through a body fluid such as in urine.

The disaccharide, or other larger sugar, is a measure of loosening of tight junctions between epithelial cells. The monosaccharide, typically rhamnose or mannitol, on the other hand is believed to cross the epithelium predominantly transcellularly and as such is said to reflect changes in surface area or as a marker of absorptive function.

The ratio of the levels of Lactulose/Rhamnose ratio in blood has been claimed to, in part, reflect damage. However, it is unclear quite what is measured and it is unlikely that it is a precise marker of severity of mucosal damage.

The present invention provides a sugar-based breath test which provides diagnostic information with regard to the functioning of the gut in an animal, including avian, subjects.

### SUMMARY OF THE INVENTION

The present invention contemplates a non-invasive sugar-based assay for assessing gut function in an animal, including avian, subject.

In one aspect, the present invention provides a method of assessing gut function in an animal subject, said method comprising:
(i) taking one or more initial breath samples;
(ii) administering to the animal subject a labeled test molecule which is acted on by a brush border enzyme in the small intestine of the animal subject to produce a product which is further catabolized to a product measurable in breath expired by the animal subject;
(iii) administering to said animal subject one or more additional test molecules(s);
(iv) taking one or more further breath samples;
(v) taking one or more body fluid samples;
(vi) ascertaining and comparing the level of labeled product in the one or more initial breath sample(s) and the further one or more breath samples and calculating the change in labeled product after ingestion of the labeled test molecule;
(vii) ascertaining the level of the one or more additional test molecules and, optionally, the labeled test molecule, in a body fluid sample; and
   interpreting the level of absorption and/or metabolism of each of the test molecules, based on the results obtained, to assess the gut function of the animal subject.

The present invention contemplates administration of a combination of either two or three test molecules (including the labeled test molecule) either concurrently or sequentially to an animal subject. The combination may in one form be the labeled test molecule, which predominantly transcellularly crosses the epithelium of the small intestine when ingested by the animal subject. The combination may in a second form be the labeled test molecule together with the non-reactive molecule, wherein the non-reactive molecule is not substantially susceptible to enzymatic cleavage or active transport in the small intestine. A yet further test substrate the permits assessment of the colon with any one of the above forms may also be contemplated.

In a preferred embodiment, the administered molecule is a sugar including a labeled sugar. However the subject invention also comprises phosphorylated sugars, oligopeptides or oligonucleotides instead of the sugars. These compounds may be unlabeled or they may be labeled with the stable isotopes ¹³C, ¹⁵N or ¹⁸O or other radioactive or non-radioactive isotopes of C, N or O. In one form this labeling may aid in the measurement in body fluids, blood, urine, saliva and breath condensate, but measurement can also be performed using PCR, RT-PCR, ELISA and mass spectrometry.

In particularly preferred embodiments of the subject invention, the labeled test sugar is a labeled disaccharide such as sucrose, which is labeled with a radioactive or non-radioactive isotope of carbon or oxygen, a useful monosaccharide is rhamnose or mannitol and the non-reactive disaccharide is lactulose. A further test substrate such as a sucralose may also be used.

The methods of the present invention have application for assessing gut function in humans, including changes in gut function associated with a wide range of human pathologies, and also assessing gut function in other animal species including livestock animals.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A list of abbreviations used herein is provided in Table 1.

TABLE 1
Abbreviations

| ABBREVIATION | Description |
|---|---|
| GI | Gastrointestinal [tract] |
| HPLC | High Performance Liquid Chromatography |
| IRMS | Infrared Mass Spectroscopy |
| L:R | Lactulose to Rhamnose ratio |
| MTX | Methotrexate |
| SBT | Sucrose Breath Test |
| S:L | Sucrose to Lactulose ratio |
| S:R | Sucrose to Rhamnose ratio |

### BRIEF DESCRIPTION OF THE FIGURES

FIG. 1 is a graphical representation showing the levels of labeled carbon dioxide expired by patients either with or without mucositis at different time period when tested by the SBT. These data demonstrate that the small intestine is damaged and depressed in terms of absorptive capacity.

FIG. 2 is a graphical representation showing the result of the lactulose and rhamnose assays on the same test subject at the same time periods and indicates that permeability is not greatly altered until about day 7 to 9.

FIG. 3 is a graphical representation showing illustrating different stages of damage which may occur to the villous surface of the GI tract in animal subjects.

FIG. 4 is a graphical representation indicating the SBT results for patients with atopic eczema compared with control subjects.

FIGS. 5A through C are graphical representations of the relationship between the SBT and permeability in aboriginal children with a damaged small intestine.

FIG. 6 is a representation of delivery of a labeled substrate via endoscopy and orally showing a different result due to the fact that a bacterial infection is localized to the stomach and not found in the duodenum. This is Helicobacter pylori and is detected using ¹³C urea.

FIG. 7 is a diagrammatic representation showing the interactions of the SBT with a series of other parameters that can be measured in other body fluids. These are based on the SBT to other sugar paradigms as outlined in Examples 1 through 4.

### DETAILED DESCRIPTION OF THE PREFERRED EMBODIMENTS

Before describing the present invention in detail, it is to be understood that unless otherwise indicated, the subject invention is not limited to specific combinations of bioassays and methodologies, or the like, as such may vary. It is also to be understood that the terminology used herein is for the purpose of describing particular embodiments only and is not intended to be limiting.

The singular forms “a”, “an” and “the” used throughout the subject specification include plural aspects unless the context clearly dictates otherwise. Thus, for example, reference to “an animal subject” includes a single subject or two or more subjects; reference to “a breath sample” or “a body fluid sample” includes a single sample, as well as two or more samples.

The present invention utilises a labeled test molecule such as a sugar either alone or in combination with other molecules (eg sugars) to either provide for a novel dual molecule test or triple molecule test. Where sugars are used, the other sugars may comprise either one or both of:
(i) a monosaccharide which predominantly transcellularly crosses the epithelium of the small intestine when ingested by said animal subject; and/or
(ii) a non-reactive disaccharide which is not substantially susceptible to enzymatic cleavage or active transport in the small intestine.

Although the present invention will henceforth be described with respect to sugars, as indicated above, molecules such as phosphorylated sugars, oligopeptides and/or oligonucleotides may also be used.

The labeled test sugar contemplated by the present invention is preferably one that is acted on by a brush border enzyme of enterocytes lining the small intestine to allow for catabolism to a product measurable in the breath expired by the test subject. Typically the sugar will may be a disaccharide, and preferably one that utilises an enzyme that is substantially not inducible and is present in a preponderance of the population desired to be tested. Two sugars are particularly preferred, being mannose and sucrose, most preferred is sucrose.

Accordingly, in one aspect, the present invention provides a method of assessing gut function in an animal subject, said method comprising:
(i) taking one or more initial breath samples;
(ii) administering to the animal subject a labeled test sugar which is acted on by a brush border enzyme in the small intestine of the animal subject to produce a product which is further catabolized to a product measurable in the breath expired by the animal subject;
(iii) administering to said animal subject one or more additional test sugar(s);
(iv) taking one or more further breath samples;
(v) taking one or more body fluid samples;
(vi) ascertaining and comparing the level of labeled product in the one or more initial breath sample(s) and the further one or more breath samples and calculating the change in labeled product after ingestion of the labeled test sugar;
(vii) ascertaining the level of the one or more additional test sugars in a body fluid sample, and optionally, ascertaining the level of the labeled test sugar in a body fluid sample; and
   interpreting the level of absorption and/or metabolism of each of the test sugars, based on the results obtained, to assess the gut function of the animal subject.

As used herein the term “gut function” refers to any aspect of the functioning of the gut and/or structural integrity of the gut. For example, the methods of the present invention may be used to provide an indication or assessment of: the absorptive capacity of the gut; the ability of the gut to absorb nutrients; the “leakiness” or permeability of the gut; the ability of the gut to hydrolyse compounds; the surface area of the gut and/or small intestine; the functional surface area of the gut and/or small intestine; the barrier function of the gut; damage to the gut; the degree of mucosal damage in the gut; damage to the villi in, inter alia, the small intestine; the villous height of the brush border of the small intestine; the presence of any disease or pathology which is associated with a change in gut function as described herein; the presence of an inflammatory condition; the presence of a infection; as well as any other aspect of gut functioning or gut structural integrity that would be evident to one of skill in the art.

In particularly preferred embodiments of the invention, the labeled test sugar is a labeled disaccharide. Even more preferably, the labeled disaccharides contemplated by the present invention include those that are substrates for enzymes in the small intestine of an animal subject which are substantially not inducible and are also present in a preponderance of the population of the animal subject. In accordance with the present invention, sucrose is identified as a particularly useful labeled test sugar, as sucrase activity is almost ubiquitous in the small intestine of animals. However, any substrate which satisfies the criteria of being the substrate of gut enzymes which are substantially not inducible and are also present in a preponderance of the population of the animal subject may be used. For example, disaccharides such as maltose or mannose may also be used as the labeled test sugar.

The sucrose or other labeled test sugar may be labeled with any number of a range of labels. Preferably, one or more atoms of the test substrate is labeled so that carbon dioxide (CO₂) ultimately produced by the catabolism of the substrate will include the label. It will thus be readily apparent that the labeled atom might be either a carbon or an oxygen. A number of known labels might be used.

A most convenient label might be the use of an isotope other than the most common isotopes, the most common isotopes being ¹²C and ¹⁶O. Preferably, because the substrate is to be ingested, the isotope used is one that emits a low energy or no radiation, and most preferably none. Accordingly, ¹³C is a particularly preferred label. ¹³C is a stable isotope, and is present in sucrose produced in certain plants (such as plants which utilise C4 photosynthesis) at sufficiently high levels to not require addition of synthetically produced ¹³C-sucrose. Where other enzymes are to be measured and ¹³C maltose or other substrate is to be measured it will be understood that the labeled isotope might need to be made synthetically. Additionally to elicit a stronger signal some synthetic sucrose may be added to the naturally enriched sucrose.

Accordingly, in particularly preferred embodiments of the present invention, the labeled test sugar which is acted on by a brush border enzyme in the small intestine of the animal subject to produce a product which is further catabolized to a product measurable in the breath expired by the animal subject is ¹³C-sucrose.

Accordingly, the product in the breath expired by said animal subject is preferably ¹³CO₂.

However, a very rapid embodiment of the SBT test would use ¹⁴C radioactive (sucrose) for measurement of ¹⁴CO₂ and such an embodiment is within the scope of the present invention. However it is preferable to use the ¹³C stable isotope as there are no impediments to its transport and use in any setting.

Accordingly, other isotopes that may also be used include ¹⁴C. ¹⁴C is used in other breath test analyses and is considered generally safe for many individuals, it is not used generally on young children and women of child bearing age. Another common form of radioisotope used is ¹⁸O.

It will be understood that as with other uses of labeled compound, the extent of labeling need only be measurably different to that which occurs naturally. With ¹³C-sucrose the abundance in, for example, cane sugar is about 11-12 atom 0/00, and a measurable result is achievable after ingestion of a reasonable quantity of sucrose. Should the specific activity of the sucrose be higher, then less may need to be ingested. The specific activity required can, of course, be modulated to empirically find a level that provides a convenient result.

The abundance of isotopes relative to each other is usually given on a ‘per-mill’ (0/00) scale and is defined by [(Rₛₐₘₚₗₑ−R_{std})/R_{std})×1000, where R=[¹³C]/[¹²C] for both sample and reference standard gas, whose isotopic ratio is accurately known. The reference standard for ¹³C is from Belemnite of the Pee Dee Formation in South Carolina (PDB standard), with ¹³C/¹²C=11237.2±60×10⁻⁶.

Other forms of labeling of atoms might also be used and the present invention encompasses such labeling.

The mechanism of the labeled sucrose breath test (also referred to as the “SBT”) is more fully set out in co-pending international application PCT/AU02/01666 (published as WO 03/048765), which specification is incorporated herein by reference. This assay gives a measure of the level of activity of the brush border enzyme and will thus give a measure of the state of the brush border, for example whether the cumulative surface area is somewhat diminished or whether the enterocytes are damaged. However, this assay alone does not give a particular measure of the permeability of the intercellular spaces, permeability of tight junctions between the enterocytes, or to put another way, the ‘leakiness’ of the gut.

Use of the SBT in concert with measures of permeability of different sugars in the intestine allows both qualitative and also quantitative indices to be generated. These provide the means to screen for the type of damage, particularly with respect to region of the gut affected, and also to assess the severity of the damage, being a quantitative measure. Thus interpretation of the combined tests offer two levels of assessment. Further, they also provide a functional component of absorptive function which can be compared with the measure of a physical loss in surface area as measured by sugar (rhamnose) appearance in the blood or urine. It is necessary that the villous height can be close to normal to provide optimal absorption of nutrients. Reduction of this results in significantly diminished uptake eg Coeliac disease. The qualitative interpretation of the level of the SBT with respect to the ratios of sucrose/lactulose (S:L), sucrose/rhamnose (S:R) and lactulose/rhamnose (L:R), are set out in, inter alia, Example 3.

The present invention contemplates administration of a combination of either two or three test sugars either concurrently or sequentially to an animal subject. The combination may in one form be the labeled test sugar together with a monosaccharide. The combination may in a second form be the labeled test sugar together with the non-reactive disaccharide. The combination in a third form may be the labeled test sugar, the monosaccharide and the non-reactive disaccharide. It will be understood that preferred forms of these combinations will include the preferred forms of the individual sugars referred to above. A yet further test substrate the permits assessment of the colon with any one of the above forms may also be contemplated. The yet further test substrate may be sucralose

Therefore, in another aspect, the present invention provides a method of assessing gut function in an animal subject, said method comprising:
(i) taking one or more initial breath samples;
(ii) administering to the animal subject a labeled test sugar which is acted on by a brush border enzyme in the small intestine of the animal subject to produce a product which is further catabolized to a product measurable in the breath expired by the animal subject;
(iii) administering to said animal subject an additional monosaccharide which predominantly transcellularly crosses the epithelium of the small intestine when ingested by said animal subject;
(iv) taking one or more further breath samples;
(v) taking one or more body fluid samples;
(vi) ascertaining and comparing the level of labeled product in the one or more, initial breath sample(s) and the further one or more breath samples and calculating the change in labeled product after ingestion of the labeled test sugar;
(vii) ascertaining the level of the monosaccharide in a body fluid sample, and optionally, ascertaining the level of the labeled test sugar in a body fluid sample; and
   interpreting the level of absorption and/or metabolism of each of the test sugars, based on the results obtained, to assess the gut function of the animal subject.

The absorption rate of the monosaccharide which predominantly transcellularly crosses the epithelium of the small intestine when ingested by said animal subject reflects changes in the surface area of the small intestine and/or represents a measure of absorptive function.

In particularly preferred embodiments of the invention, monosaccharide is rhamnose or mannitol.

In a preferred embodiment of this aspect of the invention, the labeled disaccharide is labeled sucrose and said monosaccharide is rhamnose. In a particularly preferred embodiment the labeled disaccharide is ¹³C-sucrose.

In yet another aspect, the present invention provides a method of assessing gut function in an animal subject, said method comprising:
(i) taking one or more initial breath samples;
(ii) administering to the animal subject a labeled test sugar which is acted on by a brush border enzyme in the small intestine of the animal subject to produce a product which is further catabolized to a product measurable in the breath expired by the animal subject;
(iii) administering to said animal subject an additional non-reactive disaccharide which is not substantially susceptible to enzymatic cleavage or active transport in the small intestine;
(iv) taking one or more further breath samples;
(v) taking one or more body fluid samples;
(vi) ascertaining and comparing the level of labeled product in the one or more initial breath sample(s) and the further one or more breath samples and calculating the change in labeled product after ingestion of the labeled test sugar;
(vii) ascertaining the level of the non-reactive disaccharide in a body fluid sample and optionally, ascertaining the level of the labeled test sugar in a body fluid sample; and
   interpreting the level of absorption and/or metabolism of each of the test sugars, based on the results obtained, to assess the gut function of the animal subject.

As the non-reactive disaccharide is not substantially susceptible to enzymatic cleavage or active transport in the small intestine, absorption of the non-reactive dissacharide may be used as a measure of loosening of tight junctions between epithelial cells in the intestine of the subject.

“Non-reactive disaccharides” suitable for use with the present invention include any disaccharide which is not substantially susceptible to enzymatic cleavage or active transport in the small intestine, but which may be absorbed between the epithelial cells of the gut when the tight junctions become loosened. In preferred embodiments of the invention, the non-reactive disaccharide is lactulose.

In a preferred embodiment of this aspect of the invention, the labeled disaccharide is labeled sucrose and said non-reactive disaccharide is lactulose. In a particularly preferred embodiment the labeled disaccharide is ¹³C-sucrose.

In yet another aspect, the present invention provides a method of assessing gut function in an animal subject, said method comprising:
(i) taking one or more initial breath sample;
(ii) administering to the animal subject a labeled test sugar which is acted on by a brush border enzyme in the small intestine of the animal subject to produce a product which is further catabolized to a product measurable in the breath expired by the animal subject;
(iii) administering to said animal subject an additional non-reactive disaccharide which is not substantially susceptible to enzymatic cleavage or active transport in the small intestine;
(iv) administering to said animal subject a monosaccharide which predominantly transcellularly crosses the epithelium of the small intestine when ingested by said animal subject;
(v) taking one or more further breath samples;
(vi) taking one or more body fluid samples;
(vii) ascertaining and comparing the level of labeled product in the one or more initial breath sample(s) and the further one or more breath samples and calculating the change in labeled product after ingestion of the labeled test sugar;
(viii) ascertaining the level of the non-reactive disaccharide in a body fluid sample and optionally, ascertaining the level of the labeled test sugar in a body fluid sample; and
   interpreting the level of absorption and/or metabolism of each of the test sugars, based on the results obtained, to assess the gut function of the animal subject.

In a preferred embodiment of this aspect of the invention, the labeled disaccharide is labeled sucrose, said monosaccharide is rhamnose and said non-reactive disaccharide is lactulose. In a particularly preferred embodiment the labeled disaccharide is ¹³C-sucrose.

The present invention further contemplates modifications and additions to the methods described herein. For example the methods of the present invention may be further modified by the administration of a further test sugar to the animal subject wherein the further test sugar may be used as an indicator of colon functioning in the animal subject. Additional test sugars may be used to assess the function of the colon in the animal subject. Typically, such a sugar is a non-reactive sugar. These sugars may be used to measure increased permeability of the colon and/or diagnose “leaky gut” syndrome, wherein an increased absorption of the test sugar, and appearance in the blood or other body fluid, is indicative of increased colon permeability.

One useful sugar for examining colon permeability is sucralose.

As used herein, the term “animal subject” refers to any animal to which the methods of the present invention may be applied. Animal species contemplated by the present invention include, but in no way are limited to humans, non-human primates, livestock animals (eg. sheep, cows, pigs, horses, donkeys and goats), companion animals (eg. cats, dogs, rabbits), laboratory animals (eg. mice, rats, rabbits, guinea pigs, hamsters), avian species (eg. poultry birds, aviary birds) reptiles and amphibians. In preferred embodiments of the invention, the life form is a laboratory animal species.

As used herein the term “performance animal” refers to any animal species which is used in competition for any purpose. For example, “performance animals” include animals used in racing, such as horses, dogs, camels, pigeons and other birds, and the like; animals used for show purposes, such as dogs, cats, cows, sheep, pigs and other livestock; hunting animals such as dogs, ferrets, eagles, hawks and the like.

In accordance with the present invention, measuring changes of levels of the one or more sugars in a body fluid sugar of the test subject may be done at one or more time intervals after ingestion of the further test sugar, and compared to a control level. This may be tested by checking the levels directly in blood, or indirectly such as in urine by known methods.

In one specific embodiment, the present invention provides a method of assessing gut function in a human subject, said method comprising:
(i) taking one or more initial breath samples;
(ii) administering a composition comprising 10 to 20 g of ¹³C-sucrose; 5 g of lactulose; 1 g of rhamnose; and 150-200 ml of water to said human subject.
(iii) taking further breath samples at about 30 minutes, about 60 minutes and about 90 minutes after administration of said composition;
(iv) taking a blood sample at about 90 minutes after administration of the composition;
(v) ascertaining and comparing the level of ¹³CO₂ in the one or more initial breath sample(s) and the further one or more breath samples and calculating the change in ¹³CO₂ after ingestion of the ¹³C-sucrose;
(vi) ascertaining the level of lactulose and/or rhamnose in the blood sample and optionally, ascertaining the level of the ¹³C-sucrose in a body fluid sample; and
   interpreting the level of absorption and/or metabolism of each of the test sugars, based on the results obtained, to assess the gut function of the animal subject.

With regard to the methods of the present invention, the levels of ¹³CO₂ in the breath samples and/or the levels of one of more particular sugars in one or more body fluids may be ascertained by any convenient means, as would be evident to one of skill in the art. As described herein, the labeled test sugar (eg. labeled sucrose) must comprise a label which is ultimately transferred to CO₂ such that the metabolism and/or absorption of the sugar may be detected. With regard to the additional test sugars, eg. lactulose, rhamnose, mannitol etc., these sugars may be used when labeled or unlabeled. With regard to these additional sugars, the presence or absence of a label simply changes the way in which these sugars may be detected in a body fluid sample. For example, the levels of sugars such as sucrose, lactulose, rhamnose, mannitol and the like and/or metabolites thereof (eg. CO₂) in a breath of body fluid sample may be measured using: IRMS (eg. for ¹³C labeled compounds), rapid scintillation counting (eg. for radiolabeled compounds), as well as a number of techniques which may be used to detect unlabeled compounds such as sensitive solid state enzyme chemistry, HPLC, and the like. However it should be noted that the present invention is in no way limited by any label which is present or absent on any one of the test sugars used in the methods described herein, nor is the present invention limited in any way by the method used to detect any sugar or metabolite thereof in a breath or body fluid sample.

Body fluid and breath samples may be taken at about 90 min after baseline administration of the test sugar(s). However, one or more body fluid samples, for example blood samples, may be taken at about 420, 360, 330, 300, 270, 240, 210, 180, 120, 115, 110, 105, 100, 100, 95, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0 minutes after administration of the test sugar(s).

Further, urine may also be used as a body fluid sample instead of blood with a collection time of about 180 to about 300 minutes after administration of one or more test molecules.

The results generated by the method of the present invention, when put together and expressed as ratios very fully describe the small intestinal functional status. “Interpretation” of these results may be performed based on the considerations presented herein and in the examples. Furthermore, as will be evident to one of skill in the art, “interpretation” of the results generated by the methods described herein may involve comparison with one or more of the results with one or more standards or controls. Any necessary standards and/or controls as well as the appropriate comparisons therewith will be readily identified by a person skilled in the art with no undue experimentation.

The sucrose breath test (SBT) is an integrated marker of small intestinal damage. As mentioned, supra, this assay is described in detail in International Patent Publication WO 03/048765, which is incorporated herein by reference. Briefly, The digestion and absorption of a ¹³C substrate, such as ¹³C-sucrose, by the small intestine and subsequent metabolism of the ¹³C products in the liver leads to the production of ¹³CO₂, which can be measured in the breath. An increase in breath ¹³CO₂ relative to baseline levels reflects the digestion and absorption of the ¹³C substrate by the small intestine. It has been shown that significantly lower levels of ¹³CO₂ are exhaled by animals with damage to the small intestine when compared to healthy animals (see International Patent Publication, WO 03/048765).

Sucrose in the blood, if elevated, is indicative of stomach and duodenal permeability and also, to some extent, small intestinal damage. If the small intestine is badly damaged, as in some cases of chemotherapy-induced mucositis, then sucrose behaves almost as the totally non-absorbale lactulose. A sucrose:lactulose ratio, depending on the relative amounts of these sugars administered, would be close to one if damage is high and very small if no significant damage is present. If paracellular permeability is high without changes in sucrose this suggests little damage. The SBT directly measures this adding certainty to any interpretation with respect to small intestinal damage. Rhamnose increases in blood should reflect both altered surface area and to some extent paracellular permeability. Again the SBT will help clarify this when interpreted with respect to lactulose levels in blood, or urine.

The method of the present invention provides a more complete analysis of gut function than is possible using the dual-sugar assays as disclosed in the prior art.

First, the data generated by the method of the present invention provide a measure of the percentage contribution of damage to or “leakiness” of the stomach and a proportion of the small intestine which is affected. If the whole small intestine is affected then the extent to which this occurs can be calculated by subtracting the percentage recovery of lactulose from the percentage recovery of sucrose, in the light of the only functional measure available, the SBT. The lower the SBT the more compromised the functional capacity and the damage. Therefore, as is evident from the data, the methods of the present invention provide a much more complete picture of compromise to the small intestine.

Second, the methods of the present invention provide the ability to assess the surface area of the small intestine in terms of its “effective” capacity or its functional patency. Again, the SBT can be factored into this measure, such that both a qualitative and quantitative interpretation can be made, non-invasively, of the degree to which the small intestine is affected, physically and functionally.

No test for small intestinal function that can measure absorptive capacity, degree of mucosal damage, leakiness and some indication of the presence of inflammation and/or infection is currently available.

Accordingly, the methods of the present invention have application in understanding and monitoring physiological states such as in athletes in training, as an index of overtraining, in pregnancy for following changes through each trimester, as an objective index of changes in the weaning period and as a nutritional indicator of intestinal function in aged care amongst other indications.

Furthermore, the methods of the present invention have application for assessing changes in gut function associated with, and/or diagnosis of a wide range of human pathologies such as: Coeliac disease, Crohn's disease, Chemotherapy-induced mucositis, Enteric infections (bacterial and viral), Atopic eczema (potentially identifying those failing to thrive), Irritable bowel syndrome, Recurrent abdominal pain in children, Failure to thrive, Malnutrition, Food hypersensitivity/allergy, Peptic ulcer disease, NSAID (non-steroidal anti-inflammatory drugs) induced gut damage.

As mentioned herein, the methods of the present invention also have application in non-human animals. For example, the use of the present invention for the assessment of gut function in performance animals (eg. horses, greyhounds), companion animals (eg. dogs, cats) and for or livestock animals production animals (eg. chickens, pigs) its use is also contemplated.

The present invention has particular relevance to the assessment of gut function in production and livestock animals, because as prophylactic antibiotics are phased out in production animals, new ways to pick up early growth faltering for better husbandry will be needed for these industries to remain globally competitive.

Three clinical settings have been investigated that are illustrative of the use of this new test and the application of the methods of the present invention to these clinical settings are presented in the Examples. In one of these clinical settings an intervention has been used.

The present invention also provides compositions which may be used in accordance with the methods of the present invention.

In one aspect, the present invention provides a composition comprising a labeled test sugar which is acted on by a brush border enzyme in the small intestine of the animal subject to produce a product which is further catabolized to a product measurable in the breath expired by the animal subject; and a monosaccharide which predominantly transcellularly crosses the epithelium of the small intestine when ingested by said animal subject; together with a pharmaceutically acceptable carrier or diluent.

In a preferred embodiment of this aspect of the invention, the composition comprises labeled sucrose and rhamnose. In a particularly preferred embodiment, the composition comprises ¹³C-sucrose and rhamnose.

In yet another aspect, the present invention provides a composition comprising a labeled test sugar which is acted on by a brush border enzyme in the small intestine of the animal subject to produce a product which is further catabolized to a product measurable in the breath expired by the animal subject; and a non-reactive disaccharide which is not substantially susceptible to enzymatic cleavage or active transport in the small intestine; together with a pharmaceutically acceptable carrier or diluent.

In a preferred embodiment of this aspect of the invention, the composition comprises labeled sucrose and lactulose. In a particularly preferred embodiment, the composition comprises ¹³C-sucrose and lactulose.

In a further aspect, the present invention provides a composition comprising a labeled test sugar which is acted on by a brush border enzyme in the small intestine of the animal subject to produce a product which is further catabolized to a product measurable in the breath expired by the animal subject; a monosaccharide which predominantly transcellularly crosses the epithelium of the small intestine when ingested by said animal subject; and a non-reactive disaccharide which is not substantially susceptible to enzymatic cleavage or active transport in the small intestine; together with a pharmaceutically acceptable carrier or diluent.

In a preferred embodiment of this aspect of the invention, the composition comprises labeled sucrose, rhamnose and lactulose. In a particularly preferred embodiment, the composition comprises ¹³C-sucrose, rhamnose and lactulose.

In preferred embodiments, the compositions of the present the present invention comprise water as a pharmaceutically acceptable carrier or diluent.

In one particularly preferred embodiment, the present invention provides a composition comprising 10 to 20 g of labeled sucrose; 5 g of lactulose; 1 g of rhamnose; and 150-200 ml of water. In an even more preferred embodiment, the composition comprises 10 to 20 g of ¹³C-sucrose; 5 g of lactulose; 1 g of rhamnose; and 150-200 ml of water.

Optionally, the compositions described herein may further comprise at least one additional test sugar which may be used to assess the function of the colon in said animal subject. Preferably, the sugar which may be used to assess the function of the colon in said animal subject is sucralose.

In a very specific embodiment, the invention includes the injestion of labeled sucrose (¹⁴C or ¹³C labeled) (10-20 g)+lactulose (5 g)+rhamnose (1 g) administered in H₂O (150-200 ml). Breath is collected at time zero and 30 min intervals up to 90 min. Blood is collected at 90 min.

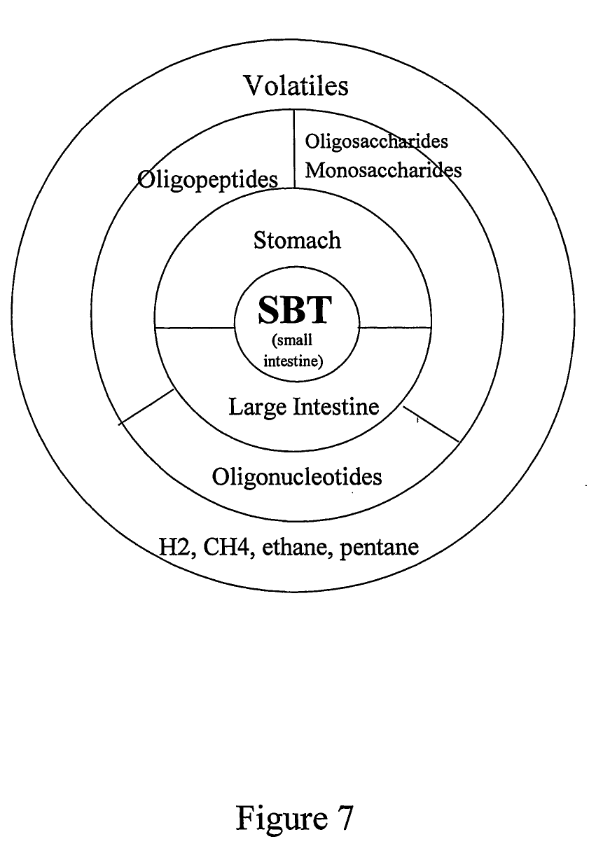

As indicated above, the methods and compositions of the present invention can be extended to incorporate the use of larger sugars including (oligosaccharides) and other macromolecules such as peptides of differing sizes and oligonucleotide fragments (RNA, DNA). All of these confer differential and regional barrier function loss when linked to the SBT levels. The SBT is the common denominator in assessing diminished barrier function throughout the gastrointestinal tract related to damage and to the pivotal absorptive capacity of the small intestine (see the diagram in FIG. 7). The paradigm using other sugars as surrogate markers of altered permeability can be extended, from a functional point of view to the different classes of molecules above eg. oligonucleotides detectable using PCR techniques allowing even greater sensitivity. These can be measured in blood, urine, saliva and breath condensate. The preferred fluids in the first instance are blood and urine followed by saliva and breath condensate. In addition phosphorylated sugars, peptides and the already phosphorylated nucleotides may form a further type of compounds and mode of measurement to be used as stable isotopically labeled (eg. ¹³C, ¹⁵N, ¹⁸O) substances. Another embodiment of the invention combines the measurement of volatile gases in the breath including H₂, CH₄, ethane and pentane at the same time as the SBT to allow for:
(i) simultaneous assessment of small intestinal transit time (H₂);
(ii) indirect assessment of an altered fermentation pattern (H₂CH₄); and
(iii) evaluation of the presence of an inflammatory process (ethane, pentane).

This allows the relationship and presence of gut inflammation to be regionalised and correlated with the severity of the inflammatory or infective events. Furthermore it allows exclusion of gut inflammation if the gut function parameters are all normal directing clinical investigations to other organ systems (eg. the lung and liver). An example of this is if ethane is elevated in the breath and the SBT is normal and no permeability is detected by presence of sugars in the blood or urine another organ system other than the gut is likely to be the source of the inflammation or infection.

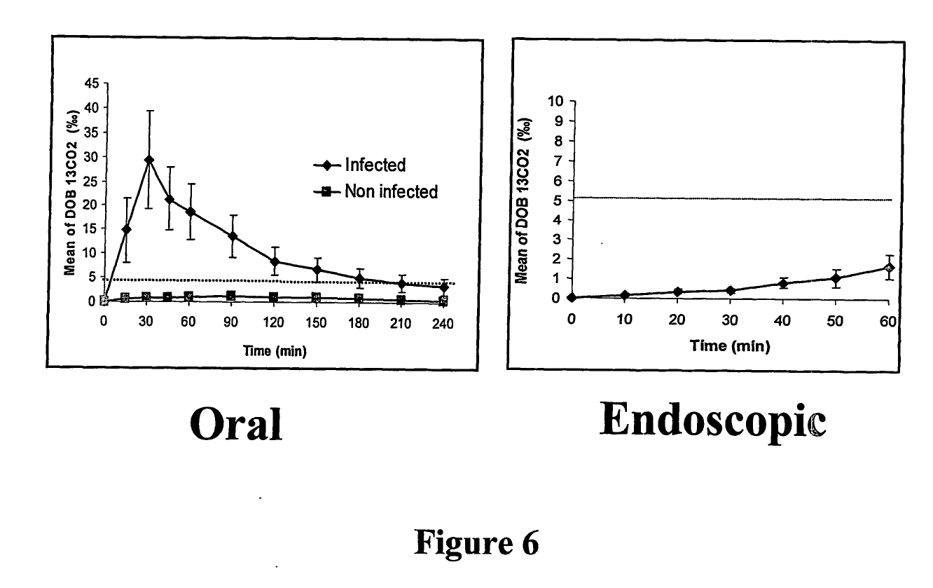

A further embodiment of the present invention is the delivery of the substrates either by catheter during endoscopic procedures (see FIG. 6 for example of this using another compound ¹³C urea), by microcapsules or by protective sheathing response to intraluminal changes such as pH and enzyme secretions. This further confers regional discrimination and enhances the precision of the estimates allowing sub-regional function to be evaluated eg. mid-ileum vs terminal ileum, proximal colon vs distal colon and monitored for damage and dysfunction.

The present invention is further described by the following non-limiting examples:

### EXAMPLE 1

### Assessment of Gut Function in Patients with Cancer and on Chemotherapy

Different chemotherapy regimens are likely to differentially damage different regions of the gut. The main area where damage occurs is probably the small intestine however the stomach, if compromised, can also contribute significantly to the side effects of an altered gut function and also severe mucositis.

For example, Methotrexate (MTX) is known to affect the small intestine more than the stomach and the colon. However, some alkylating agents will damage the stomach and others the colon.

The methods of the present invention allow discrimination between those regions affected and allow targeted preventative or repair strategies to be tested and eventually adopted.

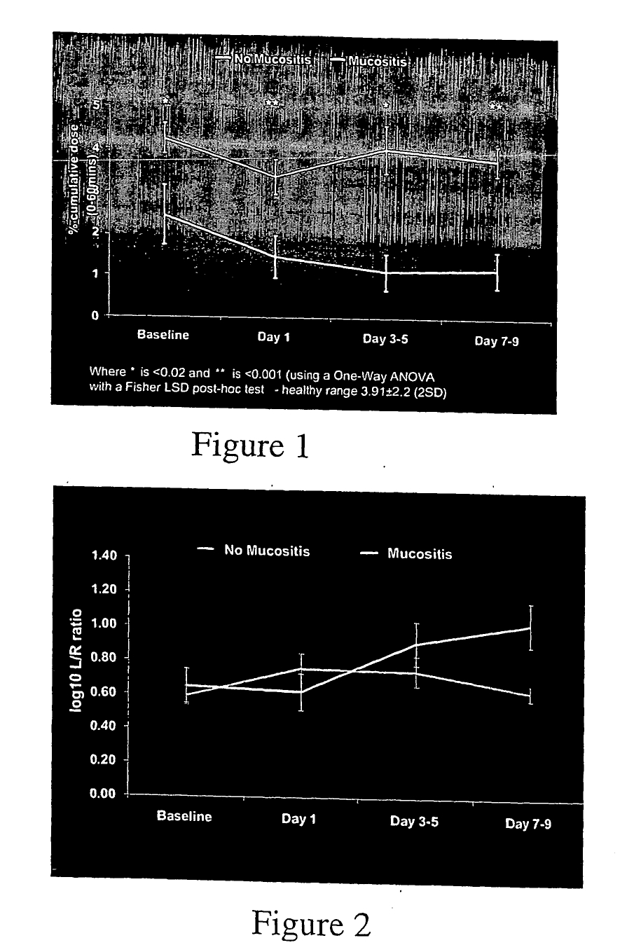

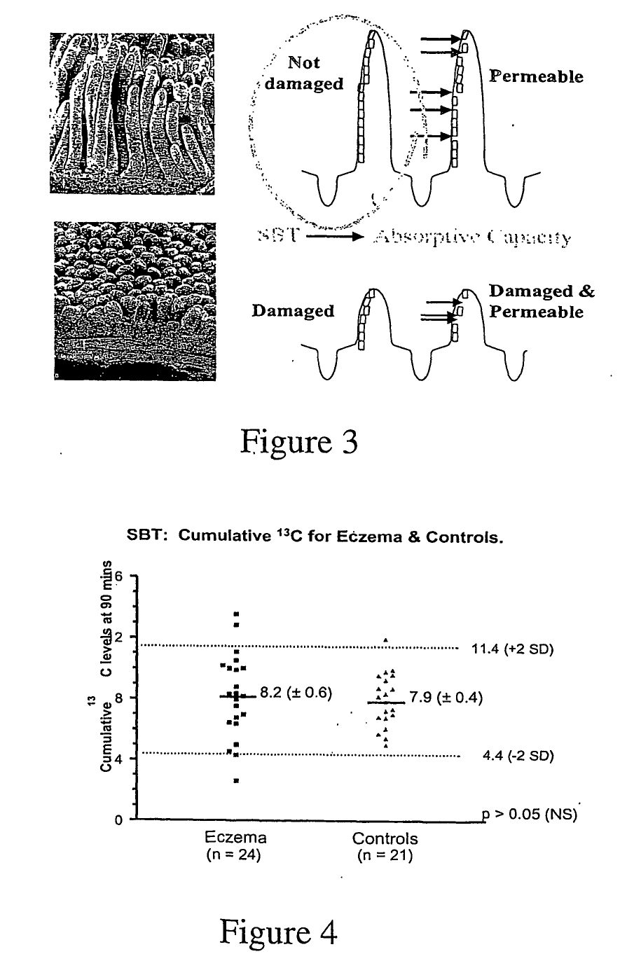

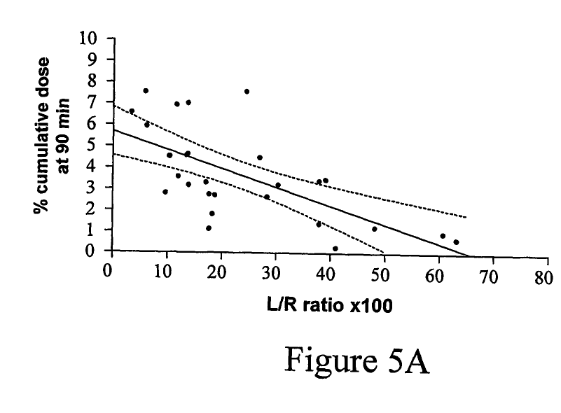

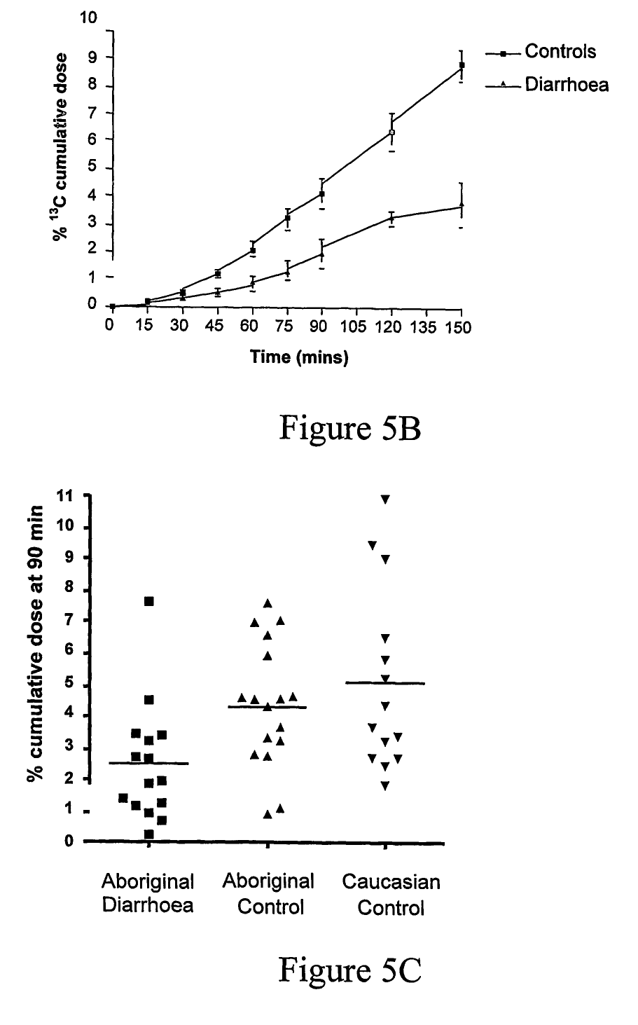

FIG. 1 shows levels of labeled carbon dioxide expired by patients either with or without mucositis at different time period when tested by the SBT as referred to herein. These data demonstrate that the small intestine is damaged and depressed in terms of absorptive capacity.

FIG. 2 is the result of the lactulose and rhamnose on the same test subject at the same time periods and indicates that permeability is not greatly altered until about day 7 to 9.

### EXAMPLE 2

### Assessment of Gut Function in Patients Atopic Eczema

The majority of patients in this category have a normal SBT but many also have increased permeability as measured by L:R ratio. This indicates that they have no damage to the villi and do not have a compromised absorptive capacity and fall into Category 3 of the proposed nomogram (see later).

An interpretation of this may be that it means that these patients do have an inflammatory event occurring and that this is sufficient and may be the cause of and/or exacerbate the intestinal permeability. It also suggests that they are at increased risk of inappropriate toxins reaching the other parts of the body (for example, the skin) and causing a flare up.

When these patients are examined individually in a larger cohort the data suggest that some do have the beginning of damage as determined by the SBT. Indeed three to four of the patients examined showed SBT levels below the normal range, although there was no correlation between the SBT and L:R ratio for the group as a whole.

FIG. 4 shows the SBT results for patients with atopic eczema compared with control subjects.

The Sucrose:Lactulose ratio (S:L ratio) in these patients may be used to select out those with an upper gut permeability, as the L:R ratio defines a whole small intestine permeability.

### EXAMPLE 3

### Qualitative Interpretation

| | | | |
|---|---|---|---|
| 1. | Normal SBT | High S:L ratio | Stomach potentially involved, |
| | | | some low grade SI damage |
| 2. | Normal SBT | Low S:L ratio | Normal |
| 3. | Normal SBT | High L:R ratio | Abnormal barrier function but |
| | | | no overt damage |
| 4. | Low SBT | High S:L ratio | Severe upper SI damage/ |
| | | | permeability |
| 5. | Low SBT | Low S:L ration | more distal SI damage |
| 6. | Low SBT | High L:R ratio | Most severe SI damage |

### EXAMPLE 4

### Quantitative Interpretation

The SBT:Rhamnose ratio may be used as a quantitative parameter. This is a more direct measure of the absorptive function and thus is indicative of the functional surface area compared to the surface area as assessed by a non- or less functional measure, eg. rhamnose passage across the mucosa.

Comparisons can also be made with the SBT:Sucrose ratio, which will determine the functional impact of small intestine damage on the extent of permeability in the upper intestine versus the SBT:Lactulose ratio, which reflects the state of the whole of small intestine. Using these parameters, the involvement of the distal small intestine compared with the proximal small intestine can be determined. This determination is particularly important in, for example, NSAID (non-steroidal anti-inflammatory drugs) induced gut damage and also in diseases such as Crohn's disease and Coeiliac disease.

### EXAMPLE 5

### Nonogram

A nomogram linking all of these measurements provides a simple way to interpret these parameters of small intestine function and will be adapted to a simple bedside measure with the appropriate small monitoring instruments. However in the first instance it is more likely that the breath and blood samples will be sent to a pathology laboratory.

Category 1

This is where severe damage has occurred and both absorptive function and permeability are affected. Patients with mucositis fit into this category.

Category 2

This is where less severe damage has occurred and where permeability may or may not be present. Some patients in the non-mucositis group fit into this category.

Category 3

This is where no apparent damage is evident as measured by the SBT but where permeability is present, eg. patients with atopic eczema.

Various features of the invention have been particularly shown and described in connection with the exemplified embodiments of the invention, however, it must be understood that these particular arrangements merely illustrate and that the invention is not limited thereto and can include various modifications falling within the spirit and scope of the invention.

1-28. (canceled)

29. A method of assessing both damage and the location of said damage to villous and non-villous regions of the gut in an animal subject, said method comprising:
(i) taking one or more initial breath samples;
(ii) administering to said subject a labeled test sugar selected from the group consisting of mannose, maltose and sucrose which is acted on by a brush border enzyme in the small intestine of said subject to produce a product which is further catabolized to a product measurable in the breath expired by said subject;
(iii) administering to said subject one or more additional test sugar(s);
(iv) taking one or more further breath samples;
(v) taking one or more body fluid samples;
(vi) ascertaining and comparing the level of said labeled product in the one or more initial breath samples and the further one or more breath samples and calculating the change in said labeled product after ingestion of said labeled test sugar;
(vii) ascertaining the level of said one or more additional test sugar(s); and
determining the relative ratios of absorption of said one or more additional test sugar(s) and said labeled test sugars which provides an indication of damage and the location of said damage to said location selected from the group consisting of the stomach and upper gut and the small intestine.

30. The method of claim 29 wherein said sucrose is ¹³C-sucrose.

31. The method of claim 29 wherein said one or more additional test sugar(s) is a monosaccharide.

32. The method of claim 31 wherein said monosaccharide predominantly transcellularly crosses the epithelium of the small intestine when ingested by said subject.

33. The method of claim 32 wherein said one or more additional test sugar(s) is selected from the group consisting of rhamnose, mannitol and lactulose.

34. The method of claim 29 wherein said one or more additional test sugar(s) is a non-reactive disaccharide which is not substantially susceptible to enzymatic cleavage or active transport in the small intestine.

35. The method of claim 29 wherein said one or more additional test sugar(s) is a sugar which may be used to assess the function of the colon in said subject.

36. The method of claim 35 wherein said one or more additional test sugar is sucralose.

37. The method of claim 29 wherein said labeled test sugar is selected from the group consisting of mannose, maltose and sucrose and said one or more additional test sugar(s) is a monosaccharide.

38. The method of claim 37 wherein said labeled sugar is labeled sucrose and said monosaccharide is rhamnose.

39. The method of claim 38 wherein said labeled sucrose is ¹³C-sucrose.

40. The method of claim 29 wherein said labeled test sugar is selected from the group consisting of mannose, maltose and sucrose and said one or more additional test sugar(s) is a non-reactive disaccharide.

41. The method of claim 40 wherein said labeled test sugar is labeled sucrose and said non-reactive disaccharide is lactulose.

42. The method of claim 41 wherein said labeled sucrose is ¹³C-sucrose.

43. The method of claim 29 wherein said labeled test sugar is selected from the group consisting of mannose, maltose and sucrose and said one or more additional test sugar(s) is selected from the group consisting of a monosaccharide and a non-reactive disaccharide.

44. The method of claim 43 wherein said labeled sugar is labeled sucrose, said monosaccharide is rhamnose and said non-reactive disaccharide is lactulose.

45. The method of claim 44 wherein said labeled sucrose is ¹³C-sucrose.

46. The method of claim 29 wherein the product in the breath expired by said subject is labeled CO₂.

47. The method of claim 46 wherein said CO₂ is ¹³CO₂.

48. The method of claim 29 wherein said subject is selected from the group consisting of a human subject, a livestock animal, a production animal, a companion animal, and a performance animal.

49. The method of claim 29, further comprising ascertaining the level of said labeled test sugar in a body fluid sample.

50. The method of claim 49, wherein said body fluid is selected form the group consisting of blood and urine.

51. A method of assessing the general state of villous health in a human subject, said method comprising:
(i) taking one or more initial breath samples;
(ii) administering a composition comprising 10 to 20 g of ¹³C-sucrose; 5 g of lactulose; 1 g of rhamnose; and 150-200 ml of water to said human subject.
(iii) taking further breath samples at about 30 minutes, about 60 minutes and about 90 minutes after administration of said composition;
(iv) taking a blood sample at about 90 minutes after administration of said composition;
(v) ascertaining and comparing the level of ¹³CO₂ in the one or more initial breath sample(s) and the further one or more breath sample(s) and calculating the change in ¹³CO₂ after ingestion of said ¹³C-sucrose;
(vi) ascertaining the level of lactulose and rhamnose in the blood sample; and wherein
determining the relative ratios of absorption of lactulose to rhamnose indicates damage to the stomach and upper gut and a decrease in the level of absorption of sucrose indicates damage in the small intestine.

52. The method of claim 51 further comprising ascertaining the level of ¹³C-sucrose in the blood.
